(19) United States
(12) Patent Application Publication
(10) Pub. No.: US 2016/0178637 A1
(43) Pub. Date: Jun. 23, 2016

(54) DETECTION OF ALPHA, BETA-DICARBONYL COMPOUNDS WITH FLUOROGENIC PROBES

(71) Applicant: THE UNIVERSITY OF WARWICK, Coventry (GB)

(72) Inventors: Paul Thornalley, Coventry (GB); Naila Rabbani, Conventry (GB)

(21) Appl. No.: 14/911,219

(22) PCT Filed: Aug. 9, 2013
(86) PCT No.: PCT/GB2013/052141

§ 371 Date: Feb. 9, 2016

(30) Foreign Application Priority Data
Sep. 10, 2012 (GB) 1216122.0

(51) Int. Cl. G01N 33/64; G01N 33/50; G01N 33/58

(52) U.S. Cl. CPC G01N 33/64; G01N 33/582; G01N 33/502; G01N 2800/323; G01N 2800/042; G01N 2800/044; G01N 2800/7042

(57) ABSTRACT

The disclosure relates to a fluorescence based assay for the detection of alpha, beta-dicarbonyl containing compounds, for example alpha-oxoaldehyde compounds such as glyoxal, methylglyoxal, hydroxypyruvaldehyde, erythrosone, 3-deoxy-erythrosone, ribosone, 3-deoxyribosone, glucosone, 3-deoxyglucosone and stereoisomers thereof and butan-2,3-dione that uses a fluorogenic probe which is stable under ambient conditions. The fluorogenic probes comprise a 1,2-diaminophenyl moiety. The diaminophenyl probes yield a 20-fold increase in quantum yield when they are a derivative of fluorescein (DAF), rhodamine (DAR), BODIPY or cyanine. The assay is preferably carried out at pH 4-8. Corresponding screening methods for modulators of alpha, beta-dicarbonyl compound concentrations are also disclosed.

The disclosure relates to a fluorescence based assay for the detection of α,β-dicarbonyl containing compounds, for example α-oxoaldehyde compounds such as methylglyoxal, glyoxal and 3-deoxyglucosone, in a sample using fluorogenic probes comprising a 4,5-diaminofluorescein, 4,5-diaminorhodamine, 3,4-diaminophenyl boron dipyrromethene or o-diaminocyanine moiety.

### BACKGROUND TO THE INVENTION

The dicarbonyl, methylglyoxal (MG), is a highly cytotoxic metabolite formed mainly from the degradation of triosephosphate intermediates of glycolysis. It is a minor product formed by spontaneous degradation of triosephosphates in mammalian metabolism—accounting for approximately 0.1% triosephosphate flux, increasing in ageing and disease, and may account for much higher triosephosphate flux in microbial metabolism where MG is from enzymatically from dihydroxyacetonephosphate by methylglyoxal synthase. MG is a potent glycating agent modifying mainly arginine residues in proteins and deoxyguanosine residues in DNA. Modifications by MG can lead to loss of function of the protein and result in DNA strand breaks and mutations at chromosomal hotspots. In physiological systems MG is mainly metabolised by the glyoxalase system comprising glyoxalase 1 (Glo1) and glyoxalase 2 (Glo2) and a catalytic amount of glutathione. Glo1 catalyses the catalyses the conversion of the hemithioacetal formed non-enzymatically from MG and reduced glutathione (GSH) to S-D-lactoylglutathione. S-D-Lactoylglutathione is then hydrolysed to D-lactate and GSH by Glo2, regenerating GSH consumed in the Glo1-catalysed reaction—FIG. 1. The glyoxalase system therefore controls and regulates the exposure of cells to MG, glyoxal and related reactive α-oxoaldehyde substrates of Glo1. Despite this detoxification system around 1-5% of proteins and approximately 1-10 adducts per 10⁶ nucleotides in DNA contain MG in vivo [1]. Increased protein modifications by MG, linked to both elevated formation of MG and down regulation of Glo1 expression, are associated with ageing and development and progression of disease—particularly vascular complications of diabetes, renal failure, critical illness, cardiovascular disease, neurological and certain mood affective disorders and arthritis [1].

The recognition that MG and Glo1 is involved in development and progression of disease led to the exploration of compounds comprising inducers of Glo1 expression potentially useful for the treatment of vascular disease occurring in diabetes and renal failure, and other disease, and to the development of functional foods for healthy ageing—including the maintenance of metabolic and vascular health [2,3]. Additionally, medicaments containing Glo1 inhibitors are being developed for the treatment of tumours where Glo1 overexpression is associated with multi-drug resistance, and similar application for treatment of malaria and other microbial infection-related disease [4]. These applications, particularly when associated with requirement for high throughout measurement for screening of large numbers of samples for diagnosis or compound library screening, require procedures for facile detection and quantitation of MG and related Glo1 substrates in physiological systems or solutions under physiological conditions. An additional advantage would be technology enabling imaging of MG concentration to visualize and quantify spatial variation in MG concentration in cells and tissues under physiological conditions and other materials under ambient conditions.

α-Oxoaldehyde compounds such as MG and glyoxal are not chromophoric nor fluorescent and therefore are not readily detectable. Chemical derivatisation of α-oxoaldehydes is usually essential for assaying to high and adequate sensitivity. The concentrations of MG and related α-oxoaldehydes in physiological samples may be easily overestimated as the sample preparation and derivatisation process permits formation of MG and related α-oxoaldehydes through the degradation of monosaccharides, glycated proteins and glycolytic intermediates. Current analytic methods of MG and related α-oxoaldehyde levels in a sample by gas or liquid chromatography coupled to a mass spectrometer require often laborious processing steps including derivatisation of the sample with 1,2-diaminobenzene. The numerous processing steps, inherent low physiological concentrations of MG and related α-oxoaldehydes in the sample (0.1-5 μM) and sub-optimal analytical methods may result in a 10-1000 fold higher estimation of sample concentration. Additionally, these methods are unsuitable for an accurate high throughput screening approach and real time monitoring.

There is therefore a need to develop fast, sensitive and quantitative assays for MG and related α-oxoaldehyde which are appropriate for use in biological samples.

### SUMMARY OF THE INVENTION

The present inventors have surprisingly found that screening for MG and other α-oxoaldehydes can be achieved using a fluorogenic probe which is stable under ambient conditions and comprises a 1,2-diaminophenyl binding group. Such fluorophores typically have a high fluorescence intensity, making them appropriate for detection of the low levels of MG which are found in biological samples. Further, the probes are stable under ambient conditions and thus can be used to detect MG and other α-oxoaldehydes under physiological conditions. This makes the probes appropriate for detection of MG and other α-oxoaldehydes in biological samples, in cell cultures and in living tissue.

The present invention therefore provides a method for the detection of α,β-dicarbonyl compounds in a sample comprising the steps:

i) forming a preparation comprising (a) a fluorogenic probe comprising a 1,2-diaminophenyl moiety, the probe being stable under ambient conditions; and (b) a sample to be tested for the presence of one or more dicarbonyl groups;
ii) reacting the preparation to provide a derivatised detectable fluorescent product; and
iii) detecting the fluorescent product, typically by excitation/emission fluorimetry;
wherein the method is carried out at from pH 4 to 8.

Typically, the specific fluorescence of MG or other dicarbonyl adduct with the probe should be sufficient to detect and quantify lower limit concentrations of MG and related dicarbonyls in 20 to 100 μl physiological fluids and/or 10-20,000 cells, equivalent to 10 to 100 fmol fluorescence adduct in multi-well microplate-based fluorescent readers or similar devices. This is specific fluorescence similar to that of the well-known fluorophore fluorescein, thus making the fluorogenic probe useful in the detection of low concentrations of α-oxoaldehydes. This is typically achieved, for example, using a fluorogenic probe having at least a 20-fold increase in quantum yield on derivatisation with MG (or other dicarbonyl). Fluorescence quantum is defined as the ratio of the number of photons emitted by the fluorophore to the number absorbed. It is conveniently determined as relative quantum yield by comparing by the intergrated emission intensities of a test and reference compound of known quantum yield at the same concentration (Lakowicz, J R (2006) Principles of Fluorescence Spectroscopy, 3^{rd} edn., Academic Press, New York). Such high intensity fluorogenic probes include those comprising a diaminofluorescein (DAF) moiety, a diaminorhodamine (DAR) moiety, a diaminophenyl boron dipyromethane (BODIPY) moiety or a diaminocyanine moiety or a functional derivative thereof. All of these materials have been found by the present inventors to be stable, highly fluorescent materials having an easily measurable change in their fluorescence emission characteristics on binding to α-oxoaldehydes such as MG.

In particular the present disclosure relates to a novel approach for rapid detection, quantification and imaging of MG and related α,β-dicarbonyl compounds involving derivatization with fluorogenic probes containing a 4,5-diaminofluorescein (DAF-2), 4,5-diaminorhodamine (DAR), 3,4-diaminophenyl boron dipyrromethene (BODIPY) or o-diaminocyanine moiety.

The technique described herein allows accurate analysis of physiological samples for α,β-dicarbonyl content via fluorimetry, fluorescence microscopy, flow cytometry and related techniques suitable for a high throughput approach (multi-well microplate and related techniques). In addition α-oxoaldehyde compounds such as methylglyoxal and related α-oxoaldehydes are known diagnostic markers of a number of conditions such as type 1 and type 2 diabetes, complications associated with diabetes such as kidney disease, retinal disease, peripheral nerve disease, cardiovascular disease, cerebrovascular disease (stroke) and cataract, atherosclerosis, hypertension, rheumatoid arthritis and osteoarthritis and renal failure with or without dialysis treatment. The disclosure therefore also relates to a method of diagnosing patients suspected of having or having a predisposition to such diseases. The detection of α-oxoaldehydes as a diagnostic biomarker is difficult due to very small concentrations in the circulation. There is therefore a need to develop fast, sensitive and quantitative assays to measure α-oxoaldehydes in isolated biological samples. Detection of α-oxoaldehyde contaminants in dialysis fluids for peritoneal dialysis and haemodialysis, other clinical products, foodstuffs and beverages are also envisaged.

The emerging interest in identifying compounds which decrease or increase in the concentrations of α-oxoaldehydes for therapeutic applications requires technology that is adaptable for screening libraries of chemical compounds in high throughput analysis. The fluorescent characteristics of the fluorogenic probes described herein, in particular, 4,5-diaminofluorescein, 4,5-diaminorhodamine or 3,4-diaminophenyl boron dipyrromethene, when bound as an adduct with α-oxoaldehydes, facilitates dicarbonyl imaging in the visible wavelength range. Use of similar o-diamino fluorogenic probes with near infrared fluorescence, such o-diaminocyanines, will facilitate surface detection of MG levels in vivo to 1-2 cm tissue depth in living animals [5].

### DESCRIPTION OF THE FIGURES

FIG. 1 is a schematic diagram of the enzyme activity of glyoxalase 1 and 2;

FIG. 2 illustrates fluorescence emission and excitation spectra of DAF-2MG. Key: Dashed line—DAF-2 only, solid line—50 μM DAF2 incubated for 1 h with 1 mM MG. Left-hand side, low wavelength range—emission spectra; right-hand side, high wavelength range—emission spectra;

FIG. 3 illustrates the reaction of 4,5-diaminofluorescein with methylglyoxal to form fluorescent MG-DAF-2 isomeric adducts;

FIG. 4 illustrates the increase in fluorescence of 4,5-diaminofluorescein with time

FIG. 5 illustrates the increase in fluorescence of 4,5-diaminofluorescein in relation to MG concentration;

FIG. 6 illustrates fluorescence imaging of MG in isolated human leukaemia using of 4,5-diaminofluorescein;

FIG. 7 illustrates fluorescence emission and excitation spectra of DAF-2MG. Key: Dashed line—DAF-2 only, solid line—50 μM DAF2 incubated for 1 h with 10 μM MG. Left-hand side, low wavelength range—emission spectra; right-hand side, high wavelength range—emission spectra;

FIG. 8 illustrates the kinetics of the reaction of fluorogenic probes with methylglyoxal and the kinetics assay of dicarbonyl concentration. DAF-2 and methylglyoxal. a.-c., Reaction of methylglyoxal with DAF-2 in 100 mM sodium phosphate buffer, pH 7.4 and 37° C. a. Reaction time course of 10 μM DAF-2 with 10 μM MG. Data are mean±SD (n=4). b. Dependence of initial rate of reaction on DAF-2 concentration, 2-20 μM, with 20 μM MG. c. Dependence of initial rate of reaction on MG concentration, 10-100 μM, with 10 μM DAF2. d.-f., Reaction of methylglyoxal with DAF-2 in 100 mM ammonium acetate buffer, pH 4.8 and 37° C. d. Reaction time course of 10 μM DAF-2 with 10 μM MG. Data are mean±SD (n=4). e. Dependence of initial rate of reaction on DAF-2 concentration, 2-20 μM, with 20 μM MG. f. Dependence of initial rate of reaction on MG concentration, 10-100 μM, with 10 μM DAF2. NB curvilinear dependence at high MG concentration due to rapid rate of reaction and rate of dehydration of MG becomes influential at high MG concentrations (reference: Thornalley P J, Yurek-George A, Argirov O K: Kinetics and mechanism of the reaction of aminoguanidine with the à-oxoaldehydes, glyoxal, methylglyoxal and 3-deoxyglucosone under physiological conditions BiochemPharmacol 2000; 60:55-65). Fluorescence measurement were made on a FLUOstar Optima microplate reader with 10 nm bandwidth filters with median excitation wavelength of 440 nm and emission wavelength of 510 nm; and

FIG. 9 illustrates a cell-based assay for detection of methylglyoxal by fluorogenic probes. a. A549 cells (9.4×10⁴ cells per cm²) in Krebs phosphate buffer in vitro incubated with 10 DAF2 μM (time of addition indicated by the arrow). Fluorescence was recorded every 5 min. Data are mean±SD (n=4). b. Colo 205 cells (9.4×10⁴ cells per cm²) in RPMI medium with 10% FBS in vitro pre-incubated with 20 μM BBGD (cell permeable Glo1 inhibitor) and then 10 μM DAR1 added (time of addition indicated by the arrow). Data are mean±SD (n=4). Fluorescence measurement were made on a FLUOstar Optima microplate reader with 10 nm bandwidth filters with median excitation wavelength of 440 nm and emission wavelength of 510 nm for DAF2 (a.) and median excitation wavelength of 545 nm and emission wavelength of 570 nm for DAR1.

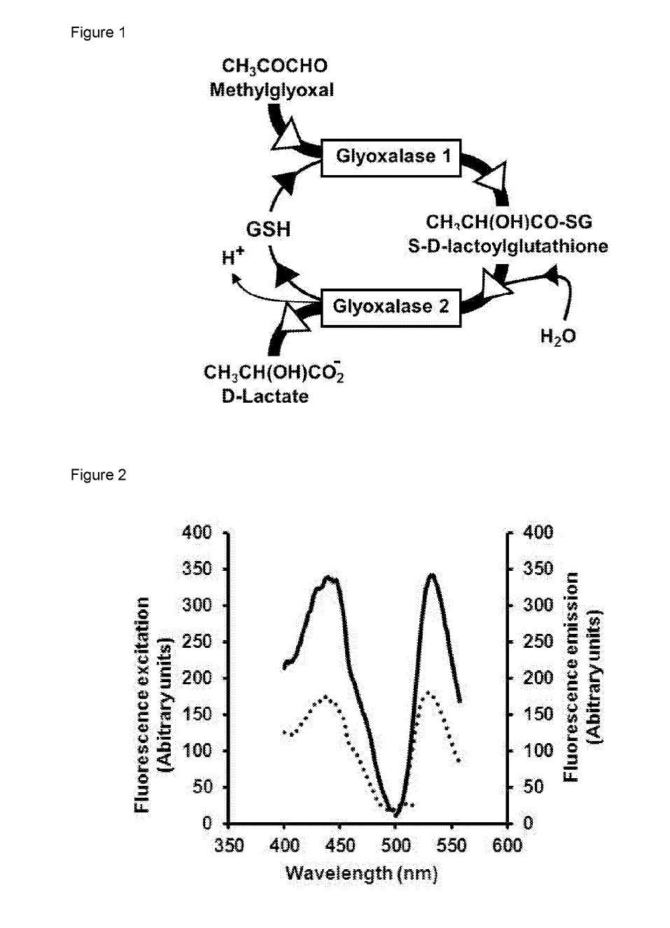

### DETAILED DESCRIPTION OF THE INVENTION

According to an aspect of the invention there is provided the use of fluorogenic probes as described herein, e.g. fluorogenic probes comprising a 4,5-diaminofluorescein, 4,5-diaminorhodamine, 3,4-diaminophenyl boron dipyrromethene or o-diaminocyanine in the detection of α,β-dicarbonyl containing compounds.

In a preferred embodiment of the invention fluorogenic probe is 4,5-diaminofluorescein, 4,5-diaminorhodamine, 3,4-diaminophenyl boron dipyrromethene or o-diaminocyanine or a functionally related derivative thereof. For example, the fluorogenic probe may be one comprising a 4,5-diaminofluorescein (DAF), 4,5-diaminorhodamine (DAR), 8-(3,4-diaminophenyl)-2,6-bis(2-carboxyethyl)-4,4-difluoro-1,3,5,7-tetramethyl-4-bora-3a,4a-diaza-s-indacene (DAM BO-P(H)), or o-diaminocyanine moiety or functional derivative thereof.

In a preferred embodiment of the invention said compounds are α-oxoaldehyde compounds.

In a further preferred embodiment of the invention said α-oxoaldehyde compound is selected from the group: glyoxal, methylglyoxal, hydroxypyruvaldehyde, erythrosone, 3-deoxyerythrosone, ribosone, 3-deoxyribosone, glucosone, 3-deoxyglucosone and stereoisomers thereof and butan-2,3-dione.

In a preferred embodiment of the invention said α-oxoaldehyde compound is methylglyoxal.

According to an aspect of the invention there is provided a method for the detection of α,β-dicarbonyl compounds in a sample comprising the steps:
i) forming a preparation comprising (a) a fluorogenic probe as described herein, in particular a probe comprising a 4,5-diaminofluorescein moiety; and (b) a sample to be tested for the presence of one or more dicarbonyl groups;
ii) reacting the preparation to provide a derivatized detectable fluorescent product; and
iii) detection of the fluorescent product by excitation/emission fluorimetry.

The method is typically carried out at pH 4-8.

In a preferred method of the invention said fluorogenic probe is 4,5-diaminofluorescein, or a functionally related derivative thereof.

In a preferred method of the invention α,β-dicarbonyl compound is a α-oxoaldehyde compound.

In a preferred method of the invention said α-oxoaldehyde compound is glyoxal, methylglyoxal, hydroxypyruvaldehyde, erythrosone, 3-deoxyerythrosone, ribosone, 3-deoxyribosone, glucosone, 3-deoxyglucosone and stereoisomers thereof.

In a preferred method of the invention said sample is a biological sample.

In a preferred method of the invention said biological sample is obtained from a subject is an isolated bodily fluid.

In a preferred method of the invention said bodily fluid is selected from the group consisting of: whole blood, plasma, serum, seminal fluid, urine, lymph fluid, cerebrospinal fluid, synovial fluid, tears, sweat, amniotic fluid, saliva or expelled breath.

In an alternative preferred method of the invention said biological sample comprises a cell or tissue.

In a preferred method of the invention said cell sample comprises a cell-line.

In an alternative method of the invention said cell or tissue sample is obtained from a subject.

Preferably, said cell-line or cell/tissue sample obtained from a subject is a mammalian cell-line or a mammalian subject; preferably a human cell line or subject.

In a preferred method of the invention said method is a method of diagnosis of a subject which has or is suspected of having a disease or condition associated with elevated levels of a α,β-dicarbonyl compound.

In a preferred method of the invention said disease is type 1 or type 2 diabetes.

In an alternative preferred method of the invention said condition is a diabetic associated condition. Preferably said diabetic associated condition is kidney disease, retinal disease, disease of peripheral nerve, cardiovascular disease and stroke, or cataract.

In a further alternative method of the invention said disease is obesity, hypertension, cardiovascular disease or renal failure.

In an alternative method of the invention said disease is pathologic anxiety, schizophrenia, Parkinson's disease or Alzheimer's disease.

In a further preferred method of the invention said disease is inflammation associated with septicaemia, burns, wounding or post-surgery trauma, rheumatoid arthritis or osteoarthritis.

In a further alternative method of the invention said disease is infertility, pre-eclampsia or other reproductive disorder.

In an alternative method of the invention said physiological state is ageing.

In an alternative preferred method of the invention said sample is a clinical dialysis fluid or other thermally sterilised fluid with sugar solutes.

In an alternative preferred method of the invention said sample is a food or drink product, particularly but non-exclusively thermally processed products.

In an alternative preferred method of the invention said sample is an environmental solution or vapour or smoke condensate, particularly but non-exclusively samples of smoke condensate from burning of tobacco leaves or other materials and samples collected for environmental pollution control.

According to an aspect of the invention there is provided an agent comprising a fluorogenic probe as described herein, e.g. a fluorogenic probe comprising a 4,5-diaminofluorescein moiety, for use as an imaging agent for the detection of α,β-dicarbonyl compounds containing compounds in cells and subjects

In a preferred embodiment of the invention said fluorogenic probe is 4,5-diaminofluorescein, 4,5-diaminorhodamine, boron dipyrromethene or o-diaminocyanines or a functionally related derivative thereof.

Throughout the description and claims of this specification, the words “comprise” and “contain” and variations of the words, for example “comprising” and “comprises”, means “including but not limited to”, and is not intended to (and does not) exclude other moieties, additives, components, integers or steps. “Consisting essentially” means having the essential integers but including integers which do not materially affect the function of the essential integers.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### Fluorogenic Probes

The term flurogenic probe as used herein refers to a compound which interacts with a substrate to produce a change in the fluorescence emission properties of the compound. Typically, a fluorescent probe will interact with a substrate in such a way that the fluorescence intensity, wavelength and/or lifetime of the probe is altered in the presence of the substrate. In the present invention, the fluorogenic probe comprises a 1,2-diamino phenyl group which is capable of binding to α,β-dicarbonyl containing compounds to produce a derivatised probe (also referred to herein as fluorescent product and detectable fluorescent product). Thus, in the present invention, the fluorescence of the fluorogenic probe is altered on binding with an α,β-dicarbonyl containing compound, such that the derivatised probe has measurable fluorescent properties different from those of the underivatised probe.

The fluorogenic probe comprising a 1,2-diaminophenyl group may not exhibit any fluorescence when in the underivatised form (i.e. when the 1,2-diaminophenyl group is not bound to an α,β-dicarbonyl containing compound). This is because the 1,2-diaminophenyl group in some cases quenches the fluorescence of the probe. However, binding of the probe to an α,β-dicarbonyl containing compound causes the quenching effect to cease or be reduced, thus leading to fluorescence of the probe.

The change in fluorescence caused by binding to the α,β-dicarbonyl containing compound may be a change in intensity, wavelength or lifetime of the fluorescence, or a combination of two or more of these effects. Typically, the change in fluorescence comprises a change in intensity.

The fluorogenic probe preferably has a low quantum yield of less than 0.01, for example less than 0.008 or less than 0.006. The probe quantum yield may be, for example, at least 0.004. The quantum yield of the adduct of probe with MG (or other dicarbonyl) is preferably at least 0.2 and is, for example, up to 0.9. A probe having a quantum yield of 0.01, and a quantum yield when derivatised with MG of 0.2 has a 20-fold increase in quantum yield on derivatisation. Preferably, the increase in quantum yield on derivatisation is at least a 20-fold, more preferably at least 50-fold, yet more preferably at least 100-fold. Preferred increases in quantum yield are from 100 to 900 fold increases.

Preferred fluorogenic probes of the invention react with dicarbonyl compounds, in particular with MG, to produce adducts which have intense fluorescence. Intense fluorescence as used herein means that the probe has a specific fluorescence of at least 5-10% of that of fluorescein, preferably similar to that of fluorescein. Typically the quantum yield of the adduct with MG (or other dicarbonyl) is at least 100 fold higher than that of the probe.

The fluorogenic probes of the invention are stable under ambient conditions. Ambient conditions as used herein refers to a temperature of approximately 20° C., atmospheric pressure and a pH of approximately 7. Probes which are stable under such conditions substantially will not degrade when stored under these conditions. Thus, such probes will substantially not degrade over a period of 24 hours, or they will degrade at a rate of less than 5% by mol over 24 hours, preferably less than 1% by mol, more preferably less than 0.5% by mol. Degradation can be measured by determining the concentration of the fluorogenic probe, measuring again after 24 hours storage under ambient conditions and determining any reduction in concentration of the probe.

Degradation refers to chemical stability of the fluorogenic probe and stability of the adduct formed from reaction with MG and other dicarbonyls. In this regard it is also important that the probe and adducts do not significantly degrade to form MG and other dicarbonyls by interaction with the physiological sample, nor change significantly endogenous processes involved in formation or removal of MG and other dicarbonyls—accepting that a fraction of the dicarbonyl is consumed in the reporter assay. Therefore the probe and dicarbonyl adduct should not impact significantly on anaerobic glycolysis, lipid peroxidation, spontaneous oxidation and fragmentation of monosaccharides to dicarbonyl, activity of glyoxalase 1 (Glo1) and its cofactor reduced glutathione, and activities of aldoketo reductase and dehydrogenase isozymes that metabolise dicarbonyls in physiological samples. In one aspect, where the sample is a non-biological sample, 0.3% sodium azide is added to increase stability.

The probes described herein typically comprise a diaminofluorescein (DAF), diaminorhodamine (DAR), diaminophenyl boron dipyrromethane (BODIPY) or diaminocyanine moiety or a functional derivative thereof. For example, a 4,5-diaminofluorescein (DAF-2), 4,5-diaminorhodamine (DAR-1), 5,6-diaminorhodamine (DAR-2), 3,4-diaminophenyl boron dipyrromethane (BODIPY) or o-diaminocyanine moiety or a functional derivative thereof. A particularly preferred BODIPY moiety is 8-(3,4-diaminophenyl)-2,6-bis(2-carboxyethyl)-4,4-difluoro-1,3,5,7-tetramethyl-4-bora-3a,4a-diaza-s-indacene or a functional derivative thereof. Thus, in one aspect, the fluorogenic probe is a 4,5-diaminofluorescein (DAF-2), 4,5-diaminorhodamine (DAR-1), 5,6-diaminorhodamine (DAR-2), 8-(3,4-diaminophenyl)-2,6-bis(2-carboxyethyl)-4,4-difluoro-1,3,5,7-tetramethyl-4-bora-3a,4a-diaza-s-indacene, or o-diaminocyanine moiety or a functional derivative thereof, in particular a 4,5-diaminofluorescein (DAF-2), 4,5-diaminorhodamine (DAR-1), 8-(3,4-diaminophenyl)-2,6-bis(2-carboxyethyl)-4,4-difluoro-1,3,5,7-tetramethyl-4-bora-3a,4a-diaza-s-indacene, or o-diaminocyanine moiety or a functional derivative thereof.

The functional derivatives of the above-mentioned fluorogenic compounds include, for example, compounds which have been modified to more easily cross the cell wall. The skilled person in the art would be familiar with appropriate modifications which could be made. Examples of functional derivatives of the above mentioned compounds include those modified to comprise ester groups, for example one or more, e.g. 1, 2, 3 or 4 C₁-C₄ alkyl esters. For instance DAF can be modified to diaminofluorescein diacetate, by replacement of the hydroxyl groups with acetate groups. Alternatively or additionally, one or more, e.g. 1, 2, 3 or 4 C₁-C₈ alkyl, fluorine or trifluoromethyl groups, triphenylphosphonium (TPP) cations and/or long chain alkyl groups such as C₉-C₂₀ alkyl groups, may be present as substituents on the basic structure. Triphenylphosphonium (TPP) cation moieties may be used for probe accumulation in mitochondria (Porteous C M, et al: Biochimica et Biophysica Acta (BBA)—General Subjects 2010; 1800:1009-1017). Octadecylrhodamine derivatives may be used for probe accumulation in lysosomes (Koshkaryev A, et al Journal of Drug Targeting 2011; 19:606-614).

The fluorogenic probe comprises the 1,2-diaminophenyl group, for example it may comprise one of the fluorogenic moieties described above. The compound may be bound to further moieties, for example the probe may be bound to further moieties which will provide selectivity for particular cell types. Examples of this include conjugation of the probe to a TAT or RGD peptides for delivery into cells (Srinivasan D, et al: Conjugation to the Cell-Penetrating Peptide TAT Potentiates the Photodynamic Effect of Carboxytetramethylrhodamine. PLoS ONE 2011; 6:e17732; Mokhtarieh A A, et al Biochemical and Biophysical Research Communications 2013; 432:359-364), and conjugation to a cell permeable, nucleus localizing peptide such as VQRKRQKLMP-NH2 for cell nucleus localisation (Ragin A D, et al Chemistry & biology 2002; 9:943-948).

Alternatively, the fluorogenic probe is not bound to further entities and is diaminofluorescein (DAF), diaminorhodamine (DAR), diaminophenyl boron dipyrromethane (BODIPY) or diaminocyanine or a functional derivative thereof, the preferred DAF, DAR, BODIPY, diaminocyanine and functional derivatives being as described above. Typically, the fluorogenic probe is not a functional derivative.

Excitation fluorescence is typically measured in the range 400 to 560 nm and emission in the range of 500 to 600 nm. Where the fluorogenic probe is DAF, typically excitation fluorescence is detected at from 425 to 445, for example at 435 to 445 nm, e.g. about 441 nm, and emission fluorescence is detected between 500 and 550 nm, e.g. 500 to 540 nm, e.g. about 533 nm.

Where the fluorogenic probe is DAF-2, DAR-1 or DAR-2, typical excitation fluorescence emission fluorescence detection ranges are:

DAF-2: excitation wavelength 425-445 nm (λmax 435 nm for DAF-2—MG adduct), emission wavelength 500-540 nm (λmax 509 nm for DAF-2—MG adduct).
DAR-1: excitation wavelength 535-555 nm (λmax 545 nm for DAR-1—MG adduct), emission wavelength 565-590 nm (λmax 566 nm for DAR-1—MG adduct).
DAR-2: excitation wavelength 535-555 nm (λmax 546 nm for DAR-2—MG adduct), emission wavelength 565-590 nm (λmax 572 nm for DAR-2—MG adduct).

### Detection Method

The method described herein may be used for the detection of α,β dicarbonyl compounds, in particular α-oxo aldehydes, most preferably glyoxal, methylglyoxal, hydroxypyruvaldehyde, erythrosone, 3-deoxyerythrosone, ribosone, 3-deoxyribosone, glucosone, 3-deoxyglucosone and stereoisomers thereof. In particular it is used for the detection of methylglyoxal (MG).

The method is useful on a wide variety of samples. Since the method can be carried out under physiological conditions, the sample can be a bodily fluid or a cell or tissue sample. Particular examples of samples on which testing can be carried out include bodily fluids, for example those described herein, dialysis fluids, foods and beverages, biological samples including cell or tissue samples, as described herein.

Where a cell culture is used as the sample, the method may be used to detect α,β-dicarbonyl compounds either within or outside of the cell. For example, a functionalised fluorogenic probe such as DAF diacetate can be used which will cross the cell wall and therefore detect intracellular dicarbonyl compounds. Alternatively, using a material such as DAF provides a measurement of dicarbonyl compounds outside the cell since the charged hydroxyl groups inhibit the probe crossing the cell wall.

The method is carried out at a pH of from 4 to 8, typically a pH of at least 5 or at least 6, for example a pH of no more than 7.5. Such pH ranges allow the testing of biological samples including cell cultures. Highly acidic pH ranges, which can be used to stabilise 1,2-diaminobenzene derivatives are unsuitable in the present process. The temperature at which testing is carried out is typically no more than 40° C., for example the temperature may be about 37° C. Such temperatures are ideally suited to testing biological samples.

The method comprises forming a preparation comprising the sample to be tested and the fluorogenic probe. The amount of fluorogenic probe used will vary dependent on the concentration ranges which are to be detected in the sample. The fluorogenic probe should be provided in an excess to ensure accurate detection. In biological samples, the typical range of MG concentration is in the range of 10 nM to 10 μM, typically in the range of approximately 100 nM in blood plasma, and from 2-4 μM in cells. A typical concentration of fluorogenic probe can accordingly be selected. For example, the concentration might be at least 5 μM, for example at least 10 μM. This ensures that the fluorogenic probe is present in an excess. Typically, the maximum concentration of the probe will be about 20 μM. The use of concentrations of greater than 20 μM causes increases in background signal levels.

The preparation of fluorogenic probe and sample is reacted to enable the fluorogenic probe to be derivatized by binding of the 1,2-diaminophenyl group to the α,β-dicarbonyl, to produce a fluorescent detectable product. The reaction step typically involves allowing a time for derivatisation to be carried out prior to detection taking place. In one aspect of the invention, the reaction step is allowed to proceed for at least 30 minutes, e.g. at least one hour or at least 2 hours. In a cell culture medium, the reaction may be continued for up to 10 hours, e.g. up to 8 hours. Approximately 3 to 6 hours is preferred since this ensures full reaction to produce the derivatised product, and equilibrium to form between intracellular and extracellular dicarbonyls.

In an alternative aspect of the method, detection of the fluorescent product is carried out kinetically, whilst the reaction proceeds. Thus, for example, detection may be monitored immediately the preparation of sample and fluorogenic probe has been formed, or detection may begin shortly thereafter, for example one minute, 5 minutes or 10 minutes after formation of the preparation. Detection may be continued for as long as the reaction proceeds, for example up to 3 hours, one hour, or 30 minutes. Alternatively, detection may be continued for a shorter period of time and the MG or other dicarbonyl content determined kinetically. Thus, shorter reaction times of up to 25 minutes or up to 20 minutes may be envisaged.

Detection of the fluorescent properties of the derivatised product may be carried out by any suitable means for detecting fluorescence. For example, fluorimetry, fluorescence microscopy, flow cytometry are appropriate for use with the present invention. Fluorescence microplate reader instrumentation is appropriate where the assay is carried out on a medium to high throughput of samples using microplates.

### Uses of Detection Method in Diagnosis

There is increasing evidence of increased protein modification by MG in the progression of vascular complications of diabetes, renal failure, critical illness, cardiovascular disease, neurological and certain mood affective disorders and ageing. In some cases there has been evidence that this may be linked to increased formation of MG, as well as other factors such as down regulation of the Glo1 expression^{[1-3, 10]}. In particular, increased MG has been linked to type 1 and type 2 diabetes, complications associated with diabetes including kidney disease, retinal disease, peripheral nerve damage, cardiovascular disease, cerebrovascular disease (stroke) and cataract, atherosclerosis, hypertension, rheumatoid arthritis and osteoarthritis and renal failure with or without dialysis treatment. Increased MG is also implicated in neurological disorders, such as Alzheimer's disease, Parkinson's disease, pathological anxiety (More S S, et al ACS Chemical Neuroscience 2013; 4:330-338; Kurz A, et al Cell and Molecular Life Sci 2011; 68:721-733; Distler M G, et al J Clin Invest 2012; 122:2306-2315) and glyoxalase 1-deficiency-linked schizophrenia (Arai M, et al, Archives of General Psychiatry 2010; 67:589-597). Accumulation of methylglyoxal concentration is also implicated in the activity of clinical antitumour agents and in screening for development of Glo1 inhibitor antitumour agents—particularly for treatment of tumours with multidrug resistance linked to Glo1 overexpression (Thornalley P J, et al Seminars in Cell & Developmental Biology 2011; 22:318-325; Santarius T et al, Genes, Chromsomes and Cancer 2010; 49:711-725).

The present methods for detecting MG and similar dicarbonyl compounds are therefore useful in testing for increased α,β-dicarbonyl levels in samples from subjects suffering from, or who may be suffering from these diseases and disorders. The methods of the invention may also be used as a part of a diagnosis for such diseases and disorders.

Methods of diagnosis are also provided herein. Thus, the testing method described herein may be carried out on a sample isolated from a subject, e.g. a mammalian (for example human) subject, suffering from, or susceptible to, the herein-described diseases and disorders.

Particular diseases and disorders for which the present methods may be used include diabetes (type 1 and type 2), a diabetic associated disease which may be kidney disease, retinal disease, disease of peripheral nerve, cardiovascular disease and stroke or cataract, in particular type 1 and type 2 diabetes. Further diseases for which the present methods may be used are those described above.

Testing as a part of a diagnostic method may involve the testing of a bodily fluid sample from a subject, for example a blood, plasma, serum, seminal fluid, urine, lymph fluid, cerebrospinal fluid, synovial fluid, tears, sweat, amniotic fluid, saliva or expelled breath. Plasma, serum and urine are preferred.

Alternatively, testing may be carried out on a tissue or cell sample from a subject.

### Imaging Methods

The fluorogenic probes described herein can also be used to provide imaging of α,β-dicarbonyl levels in live materials. Thus, for example, cells for testing may be incubated with the fluorogenic probe for the time periods discussed above, i.e. typically for at least 20 or 30 minutes, e.g. at least one hour or at least 2 hours, and for up to 10 hours, e.g. up to 8 hours, for example from 3 to 6 hours. Fluorimetric imaging is then carried out to provide an image of the dicarbonyl content in the sample. Such imaging can be carried out as a part of a method of diagnosis as discussed herein.

This technique allows testing to be carried out on live cell samples, or on tissue, typically at a depth of up to 1-2 cm using appropriate fluorimetric probes (e.g. o-diaminocyanine).

### Cellular Assay

The cell based screening method of the invention is useful in the identification of compounds which may modulate the concentration of α,β-dicarbonyl compounds. Modulators of MG levels are particularly desired in the search for the treatment of the diseases associated with elevated MG as discussed above. The present invention enables cellular screening assays of test compounds to be carried out, and in particular provides a method which is suitable for high through-put screening methods.

According to a further aspect of the invention there is provided a cell based screening method to determine whether a test agent modulates the concentration of α,β-dicarbonyl containing compounds comprising the steps:
i) forming a cell culture preparation comprising a cell and an agent to be tested and optionally a fluorogenic probe as described herein, e.g. a probe comprising a 4,5-diaminofluorescein moiety;
ii) culturing said the cell culture in the presence of the test agent; and
iii) testing for the presence of α,β-dicarbonyl compounds containing compounds in said cell or cell culture medium by addition of a fluorogenic probe as described herein, e.g. 4,5-diaminofluorescein, or a functionally related derivative thereof, to form a detectable fluorescent product; and
iv) detection of said fluorescent product, e.g. by excitation/emission fluorimetry.

The fluorogenic probe used in step (i) may be a fluorogenic probe comprising a 4,5-diaminofluorescein (DAF), 4,5-diaminorhodamine (DAR), 8-(3,4-diaminophenyl)-2,6-bis(2-carboxyethyl)-4,4-difluoro-1,3,5,7-tetramethyl-4-bora-3a,4a-diaza-s-indacene (DAM BO-P(H)), or o-diaminocyanine moiety or a functional derivative thereof.

The fluorogenic probe used in step (iii) may be 4,5-diaminofluorescein, o-diaminocyanine, or a functional derivative thereof.

In a preferred method of the invention said fluorogenic probe is 4,5-diaminofluorescein, or o-diaminocyanine or a functional derivative thereof, e.g. 4,5-diaminofluorescein or a functionally related derivative thereof.

In a preferred method of the invention the detection of said fluorescent detectable product is by excitation at between 435-445 nm and emission between 500-550 nm; preferably excitation at about 441 nm and emission at about 533 nm or preferably excitation at about 435 nm and emission at about 509 nm.

Preferably said α,β-dicarbonyl containing compound is a α-oxoaldehyde compound.

In a preferred method of the invention said α-oxoaldehyde compound is selected from the group: glyoxal, methylglyoxal, hydroxypyruvaldehyde, erythrosone, 3-deoxyerythrosone, ribosone, 3-deoxyribosone, glucosone, 3-deoxyglucosone and stereoisomers thereof and butan-2,3-dione.

In a preferred method of the invention said modulation is the decrease in concentration of α,β-dicarbonyl compound production.

In an alternative preferred method of the invention said modulation is increase in concentration of α,β-dicarbonyl compound.

In a preferred method of the invention said cell based screening assay comprises a plurality of cell based assays contained in a cell culture vessel adapted for high through put screening.

In a preferred method of the invention said cell based screening assay includes an inhibitor wherein said inhibitor prevents or reduces the derivatisation of nitric oxide by 4, 5-diaminofluorescein.

“Cell culture vessel” is defined as any means suitable to contain the above described cell culture assay. Typically, an example of such a vessel is a multi-well culture dish or well insert. Multiwell culture dishes are multiwell microtitre plates with formats such as 6, 12, 48, 96 and 384 wells which are typically used for compatibility with automated loading and robotic handling systems. Typically, high throughput screens use homogeneous mixtures of agents with an indicator compound that is either converted or modified resulting in the production of a signal. The signal is measured by suitable means (for example detection of fluorescence emission by fluorescence microscopy, flow cytometry of fluorimetry) followed by integration of the signals from each well containing the cells, substrate/agent and indicator compound.

In a preferred method of the invention said cell culture vessel is adapted to co-operate with a fluorimetric plate reader.

In a preferred method of the invention said screening method includes the steps of: collating the activity data in (iii) above; converting the collated data into a data analysable form; and optionally providing an output for the analysed data.

A number of methods are known which image and extract information concerning the spatial and temporal changes occurring in cells by detection of fluorescent markers. For example, U.S. Pat. No. 5,989,835 and U.S. Pat. No. 9,031,271, both of which are incorporated by reference, disclose optical systems for determining the distribution or activity of fluorescent reporter molecules in cells for screening large numbers of agents for biological activity. The systems disclosed in the above patents also describe a computerised method for processing, storing and displaying the data generated.

Step (iii) of the cellular screening assay described herein, namely testing for the presence of dicarbonyl compounds, is typically carried out as described in detail herein.

Further discussion of screening methods is provided in the Examples below and in WO 2011/161436, the content of which is incorporated herein by reference.

Embodiments of the invention will now be described by example only and with reference to the figures.

### Example 1

### Reaction of methylglyoxal with 4,5-diaminofluorescein (DAF-2)

4,5-Diaminofluorescein (DAF-2) was a fluorogenic probe developed for the detection of nitric oxide [6]. 4,5-Diaminorhodamine (DAR) [7], 8-(3,4-diaminophenyl)-2,6-bis(2-carboxyethyl)-4,4-difluoro-1,3,5,7-tetramethyl-4-bora-3a,4a-diaza-s-indacene (DAM BO-P″) [8] and o-diaminocyanine [5] are analogous o-diamino derivatizing agents based on different intense fluorophores. DAF-2 reacts with nitric oxide to form the fluorophore triazolofluorescein [6]. For example, incubation of DAF-2 (50 μM) with the NO donor (Z)-1-[2-(2-aminoethyl)-N-(2-ammonioethyl)amino]diazen-1-ium-1,2-diolate (DETA-nonoate), 10 μM, in 10 mM sodium phosphate buffer, pH 7.4 at 37° C., for one hour gave characteristic fluorescence of triazolofluorescein—excitation and emission maximum wavelengths of 491 and 515 nm, respectively. A further metabolite that reacts with DAF-2 is ascorbic acid which forms adducts DAF-2-DHA-5008 and DAF-2-DHA-518 which are fluorescent with excitation and emission wavelength maxima of 490 and 514 nm, respectively [9].

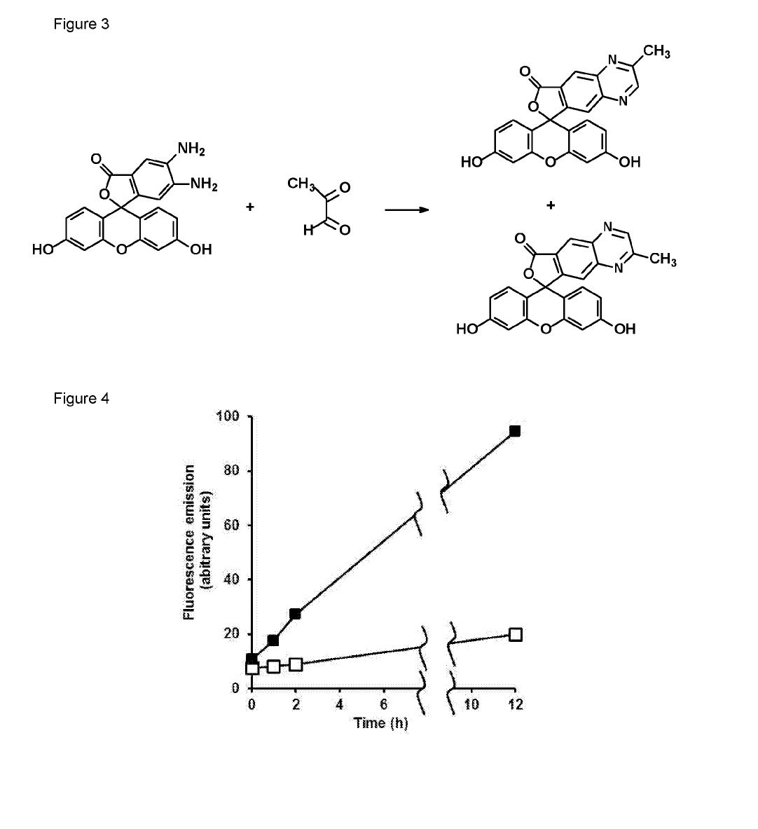

When DAF-2 (50 μM) was incubated with MG (1 mM) in 10 mM phosphate buffer at pH 7.4 at 37° C. and fluorescence spectra were taken at time 0, 1 and 2 h a different fluorescence characteristic of a isomeric mixture of MG-DAF2 adduct developed and maximised within 1 h. The excitation and emission wavelength maxima were 441 and 533 nm, respectively—FIG. 2. Mass spectrometric analysis of this compound confirmed the structure of these adducts, MG-DAF2 ((M+1)/z=MW 399.4 Da)—FIG. 3.

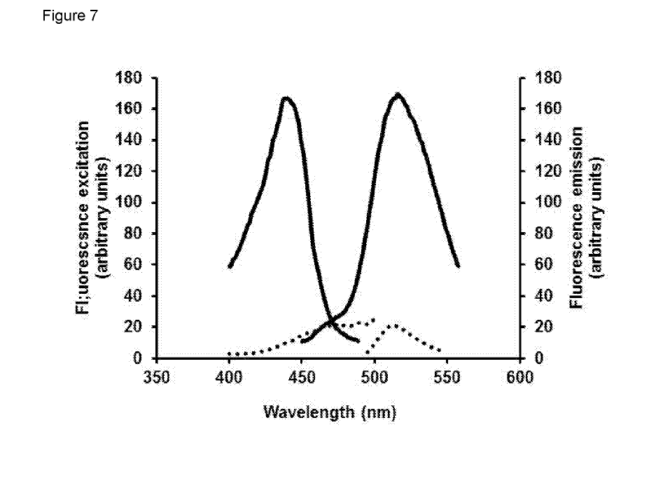

When DAF-2 (50 μM) was incubated with MG (10 μM) in 10 mM phosphate buffer at pH 7.4 at 37° C. and fluorescence spectra were taken at time 0, 1 and 2 h a different fluorescence characteristic of a isomeric mixture of MG-DAF2 adduct developed and maximised within 1 h. The excitation and emission wavelength maxima were 435 and 509 nm, respectively—FIG. 7.

### Example 2

### Dose Response Curve for the Reaction of DAF-2 with Methylglyoxal

DAF-2 (50 μM) was incubated with and without 20 μM MG in 10 mM phosphate buffer at pH 7.4 at 37° C. for 24 h. There was a marked increase in fluorescence (excitation wavelength 441 nm and emission wavelength 533 nm) over the incubation period—FIG. 4. When DAF-2 (50 μM) was incubated with 2, 5, 10, 15, and 20 μM MG in 10 mM phosphate buffer at pH 7.4 at 37° C. for 24 h and fluorescence spectra were recorded for excitation and emission wavelength maxima for MG-DAF2, 441 nm and 533 nm respectively, after 24 h the results indicate that the formation of MG-DAF2 fluorescence is proportional to initial MG concentration—FIG. 5.

### Example 3

### Fluorescence Imaging of MG in Isolated Human Leukaemia

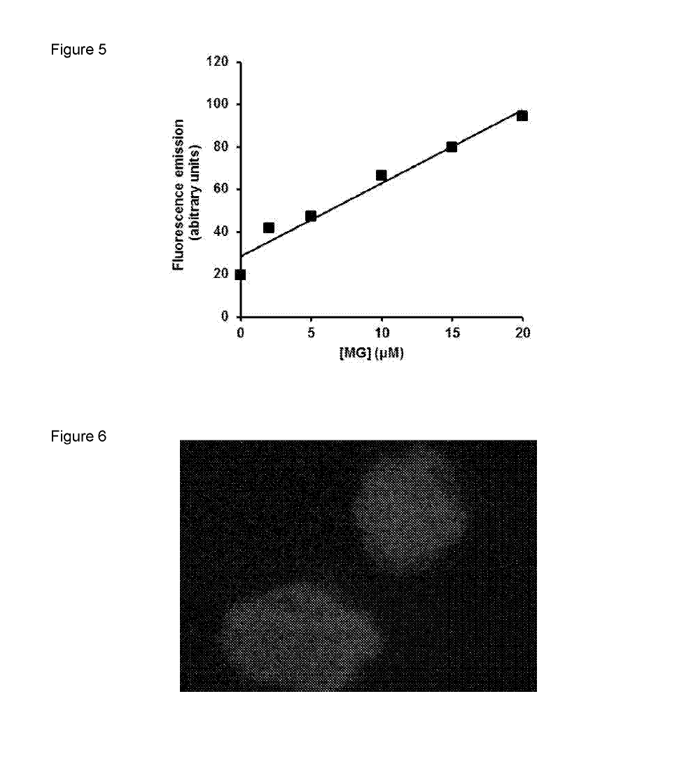

When human leukaemia 60 cells (1×10⁵ cells per ml) in RPMI 1640 with 10% fetal calf serum were incubated with DAF-2 (10 μM) for 40 min at 37° C., washed twice with phosphate buffered saline and re-suspended in fresh medium and incubated for 30-120 min, subsequent detection of fluorescence with excitation laser 457 nm, emission filter 470-500 nm gave fluorescence indicate of the DAF-2 MG adduct and hence an image of endogenous MG in HL60 cells—FIG. 6.

### Example 4

### An Example of Cell-Based Assay of Methylglyoxal Using Fluorogenic Derivatisation Methods

A549 cells are cultured in Dulbecco's Modified Eagle Medium (DMEM) medium supplemented with 2 mM L-glutamine and 10% foetal bovine serum (FBS) and NCL-H522 and Colo 205 cells are cultured in Roswell Park Memorial Institute (RPMI) medium supplemented with 10% FBS under an atmosphere of 5% CO₂ in air and aseptic conditions. Cells seeded at a density of 6×10³ cells per cm² and grown to 6×10⁴ cells per cm² (80% confluence). Cells are collected using trypsin-EDTA, neutralizing the trypsin with fresh medium, collecting the cells by centrifugation (250 g, 5 min) and countered using a hemocytometer or related technique.

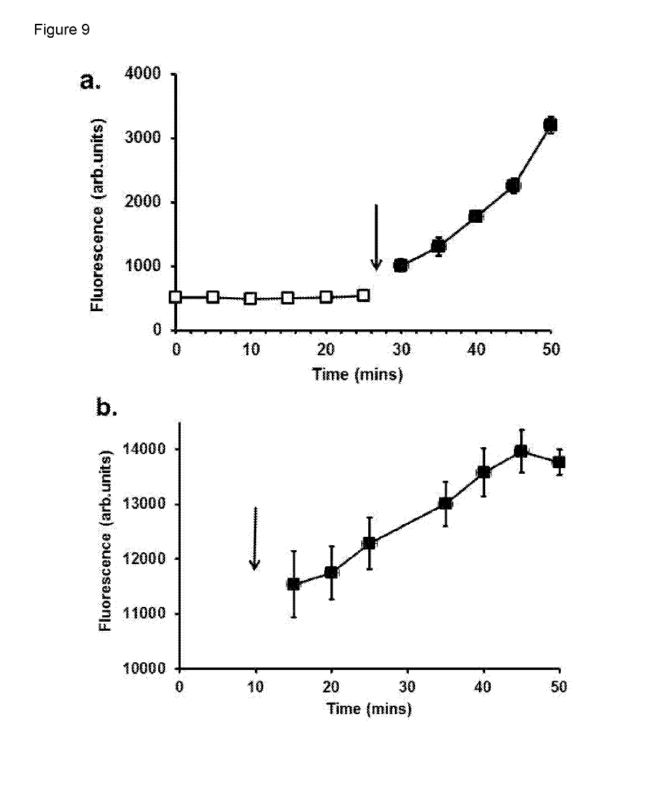

For MG assay the cells are seeded at a density of 30,000 cells per well of a black 96 well tissue culture plate (ca. 9.4×10⁴ cells per cm²) in 100 μl of culture medium and incubated for 2 days. Baseline fluorescence of the cells is measured using a microplate spectrofluorimeter for, 5 measurements with 5 min intervals between readings over 20 min. The microplate is then removed from the spectrofluorimeter and 10 μM (final concentration) DAF-2, DAF2-DA or DAR-1 added. The microplate is immediately returned to the spectrofluorimeter and fluorescence recorded at time zero and every 5 min for up to 3 h. The initial rate of increase in fluorescence is directly proportional to the MG concentration in the sample—FIG. 9.

Controls include:
(i) Addition of a dicarbonyl scavenger, 500 μM aminoguanidine, 1 h prior to addition of the fluorogenic probe to block dicarbonyl-mediated response (Thornalley P J: Use of aminoguanidine (Pimagedine) to prevent the formation of advanced glycation end products. Arch Biochem Biophys 2003; 419:31-40)—negative control for dicarbonyl response;
(ii) Addition of exogenous MG (typically 10-100 μM)—postive control for dicarbonyl response.
(iii) Addition of Nω-nitro-L-arginine methyl ester (L-NAME) 3 h prior to addition of the fluorigenic probe to block nitric oxide (NO) formation and control for possible interaction of NO with the probe to produce fluorescence.
(iv) Addition of the prototype Glo1 inhibitor prodrug, S-p-bromobenzylglutathione cyclopentyl diester (BBGD), 3 h prior to addition of the fluorigenic probe to block MG metabolism by Glo1 leading to its accumulation in the sample (Thornalley P J, Edwards L G, Kang Y, Wyatt C, Davies N, Ladan M J, Double J: Antitumour activity of S-p-bromobenzylglutathione cyclopentyl diester in vitro and in vivo. Inhibition of glyoxalase I and induction of apoptosis. BiochemPharmacol 1996; 51:1365-1372)—positive control for Glo1 inhibitor screening.
(v) Addition of 10 μmol trans-resveratrol 24 h prior to addition of the fluorigenic probe to increase metabolism of MG by induction of Glo1 expression (Cheng A S, Cheng Y H, Chiou C H, Chang T L: Resveratrol Upregulates Nrf2 Expression To Attenuate Methylglyoxal-Induced Insulin Resistance in Hep G2 Cells. Journal of Agricultural and Food Chemistry 2012; 60:9180-9187)—positive control for Glo1 inducer screening.
(vi) Addition of vehicle used for probe delivery, typically 0.2% dimethylsulfoxide (DMSO) in culture medium to cells to control for any fluorescence of the vehicle—typically not significant.
(vii) Addition of the fluorogenic probe to unconditioned medium to assess MG concentration in the medium at baseline.

Reagents required:

### 1. DAF-2/DAF2-DA/DAR-1 Solution.

Probes are typically stored at −20° C. as 5 mM solutions in DMSO, made aseptic by filtration through 0.2 μm pore size sterile microspin filters. The stock is diluted in the appropriate cell culture medium to produce a working solution 40 μM and kept on ice in the dark until use.

### 2. High Purity MG Solution

Commercial methylglyoxal solution contain formaldehyde and other contaminants. High purity MG is prepared and calibrated as described (McLellan A C, Thornalley P J: Synthesis and chromatography of 1,2-diamino-4,5-dimethoxybenzene, 6,7-dimethoxy-2-methylquinoxaline and 6,7-dimethoxy-2,3-dimethylquinoxaline for use in a liquid chromatographic fluorimetric assay of methylglyoxal. Anal Chim Acta 1992; 263:137-142; McLellan A C, Phillips S A, Thornalley P J: The assay of methylglyoxal in biological systems by derivatization with 1,2-diamino-4,5-dimethoxybenzene. Anal Biochem 1992; 206:17-23). A working solution of 30 mM is prepared for dilution in appropriate cell culture medium to produce a working solution of 1 mM immediately for use (with regard to the consumption of MG by reaction with serum protein [Thornalley P J: Dicarbonyl intermediates in the Maillard reaction. Ann NY Acad Sci 2005; 1043:111-117]).

### L-NAME Solution

Stock solution of 100 mM solution and for treatment use at a final concentration of 1 mM for a 3 h pre-incubation.

### Aminoguanidine Solution

A stock solution of 5 mM aminoguanidine hydrochloride is prepared for use at a final concentration of 500 μM.

### S-p-Bromobenzylglutathione Cyclopentyl Diester

A stock solution of 100 mM BBGD in DMSO is prepared for use at a final concentration of 20 μM. BBGD is prepared and purified as described ((Thornalley P J, Edwards L G, Kang Y, Wyatt C, Davies N, Ladan M J, Double J: Antitumour activity of S-p-bromobenzylglutathione cyclopentyl diester in vitro and in vivo. Inhibition of glyoxalase I and induction of apoptosis. BiochemPharmacol 1996; 51:1365-1372).

### Example 5

### Reaction Kinetics of Fluorigenic Probes with Methylglyoxal and Kinetic Assay of Dicarbonyl Concentration

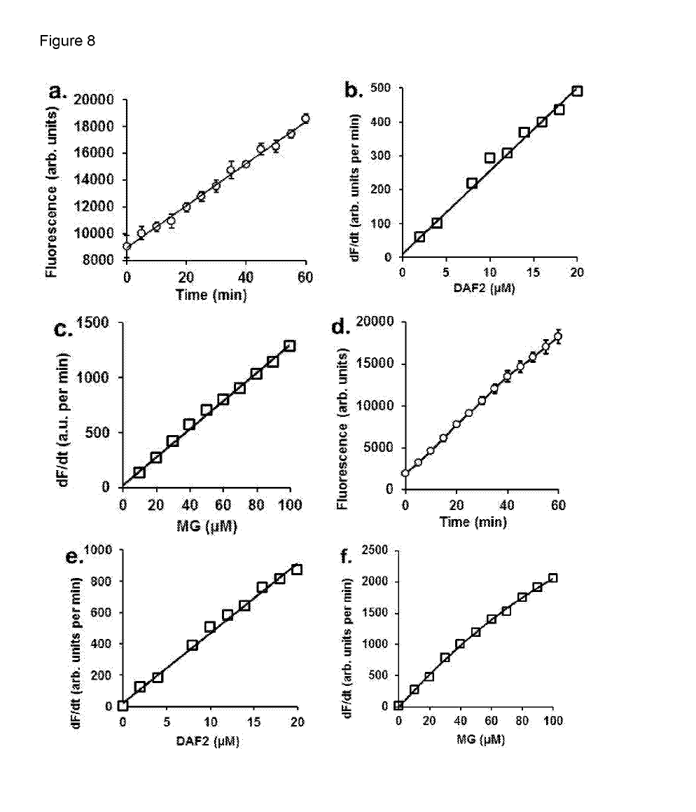

Fluorigenic probes (2-20 μM) were incubated with 20 μM MG in buffer and 37° C. and fluorescence of the MG adduct monitored for 60 min using wavelength maxima for fluorescence excitation and emission described above. MG (10-100 μM) was then similarly incubated with a fixed concentration of fluorogenic probe (10 μM) and rate of increase in fluorescence emission monitored. Buffers employed were: 100 mM sodium phosphate buffer, pH 7.4; 50 mM sodium phosphate buffer, pH 6.6—appropriate for application to the assay of glyoxalase 1 activity—see Example 6; and 100 mM sodium acetate buffer, pH 4.8—appropriate for application to the assay of dicarbonyl compounds in weakly acidic dialysis fluids. Initial rates of increase in fluorescence were deduced and plotted against initial fluorogenic probe concentration (for studies with constant MG concentration) and plotted against initial MG concentration (for studies with constant fluorogenic probe concentration)—FIG. 8. The outcome indicated that the reaction kinetics of MG with fluorogenic probes is first order with respect to fluorogenic probe concentration and MG concentration. For a fixed concentration of fluorogenic probe (typically 10 μM) the initial rate of formation of fluorescence is proportional to the methylglyoxal concentration. This forms the basis of a kinetic-based assay of MG or other dicarbonyl concentration in physiological and other samples where dicarbonyl concentration may be estimated from a 10-20 min monitoring of increase in fluorescence with fluorogenic probes added, calibrated against assay of authentic dicarbonyl standards or by a standard additional analysis protocol.

### Example 6

### Use of Fluorogenic Probes for Screening Glyoxalase 1 Inhibitors in Cell-Free System

Glo1 catalysed the conversion of the hemithioacetal of methylglyoxal and GSH formed non-enzymatically to S-D-lactoylglutathione: CH₃COCH(OH)—SG→CH₃CH(OH)CO—SG. The rate of this reaction is conveniently followed by measuring the increased in absorbance at 240 nm for the forward reaction for which the change in extinction coefficient Δε₂₄₀=2.86 mM⁻¹ cm⁻¹ (Allen R E, Lo T W C, Thornalley P J: A simplified method for the purification of human red blood cell glyoxalase I. Characteristics, immunoblotting and inhibitor studies. J Prot Chem 1993; 12:111-119). This assay is not readily transferable to high throughout assay techniques where measurement of absorbance or fluorescence emission in the visible wavelength range is preferred. Accordingly our fluorogenic probes were adapted for this purpose. The example given is that of DAF-2. Replicate samples of substrate for assay of Glo1 activity are prepared by incubation of 50 μl aliquots of 2 mM MG and 2 mM GSH in 50 mM sodium phosphate buffer, pH 6.6, for 10 min at 37° C. Glo1 (5 mU) is then added with or without prospective Glo1 inhibitor compounds and S-p-bromobenzylglutathione (known Glo1 inhibitor, as positive control). The reaction is incubated for 10 min at 37° C. where in inhibitor blanks approximately 50% of the initial MG has been converted to S-D-lactoylglutathione. Aliquots of the reaction mixtures (5 μl) are withdrawn and further reaction slowed by 20-fold dilution in 50 mM sodium phosphate buffer, pH 6.6 (95 μl). The residual MG concentration in this diluted extract is determined by the kinetic assay described above—50 μM in assays without inhibitor and 51-100 μM in assays with Glo1 inhibitor, depending on inhibitor potency.

### Example 7

### Characteristics of MG Adducts with Fluorogenic Probes, Separation of Probe and Adduct and Detection of Both by Liquid Chromatography-Tandem Mass Spectrometry

Fluorogenic probes DAF2, DAR1 and DAR2 (50 μM) were incubated with MG (50 μM) in 100 mM ammonium acetate buffer, pH 4.8, for 3 h and aliquots (50 μl) infused into the electrospray source of a Quattro Premier (Waters) mass spectrometer operated in positive ion mode to determine molecular mass of residual fluorogenic probe and MG adduct formed. Mass transitions for detection of probe and adduct were then developed for detection of both by multiple reaction monitoring (MRM). Reaction mixtures of probe and MG adduct could then be assayed for both by liquid chromatography-tandem mass spectrometry (LC-MS/MS). For LC-MS/MS the chromatographic column was octadecyl silica, 100×2.1 mm, 1.7 μm particle size (Acquity BEH, Waters). The initial mobile phase was 17.5 mM ammonium acetate, pH 4.8, with a linear gradient of methanol from 0-50% over 10 min and thereafter isocratic 50% methanol for a further 10 min. Molecular mass and positive ions for MRM detection conditions of fluorogenic probes and MG adduct (2 transitions each) are given below:

TABLE
MG adducts with fluorogenic probes, separation of probe and adduct and
detection of both by liquid chromatography-tandem mass spectrometry.

| | Retention | Molecular | Molecular | Fragment | Cone | Collision |
|---|---|---|---|---|---|---|
| Probe/ | time | mass | ion M + 1 | ions | voltage | energy |
| adduct | (min) | (Da) | (m/z) | (m/z) | (V) | (eV) |
| DAF2 | 7.9 | 362.1 | 363.1 | 191.40 | 79 | 75 |
| | | | | 316.9 | 79 | 35 |
| DAF2-MG | 8.8 | 398.1 | 399.1 | 200.20 | 89 | 84 |
| | | | | 353.10 | 89 | 39 |
| DAR1 | 13.8 | 472.3 | 473.3 | 385.3 | 65 | 46 |
| | | | | 411.3 | 65 | 46 |
| DAR1-MG | 11.0 & 11.3* | 508.3 | 509.3 | 421.3 | 53 | 48 |
| | | | | 465.3 | 53 | 30 |
| DAR2 | | 472.3 | 473.3 | 385.3 | 83 | 63 |
| | | | | 429.3 | 83 | 47 |
| DAR2-MG | 11.6 | 472.3 | 509.3 | 421.3 | 78 | 64 |
| | | | | 465.3 | 78 | 42 |

*Structural isomers were resolved chromatographically.
Other conditions for mass spectrometric detection: capillary voltage 0.40 kV, electrospray source temperature 120° C., desolvation gas 350° C., cone gas flow 146 L/h, desolvation gas flow 850 L/h, and collision cell gas pressure 4.7 × 10⁻³ mbar.

This LC-MS/MS method may also be used to quantify probe-dicarbonyl adduct levels to high sensitivity (<1 pmol).

### Example 8

### Quantitation of Methylglyoxal in Human Physiological Samples

### Example

### Determination of Dicarbonyl Concentration in Human Plasma

Aliquots of human plasma (50 μl) are incubated with fluorogenic probe in 100 mM sodium phosphate buffer, 0.3% sodium azide and 0.05-0.5 μM MG in standard additional analysis protocol. After incubation at 37° C. for 1 h the fluorescence of reaction mixtures, probe only, plasma only and blank controls is recoded and the plasma concentration of MG is deduced.

### REFERENCES

1. Rabbani N, Thornalley P J. (2012) Methylglyoxal, glyoxalase 1 and the dicarbonyl proteome. Amino Acids 42: 1133-1142.
2. Xue M, Rabbani N, Momiji H, Imbasi P, Anwar M M, Kitteringham N R, Park B K, Souma T, Moriguchi T, Yamamoto M, Thornalley P J. (2012) Transcriptional control of glyoxalase 1 by Nrf2 provides a stress responsive defense against dicarbonyl glycation. Biochem J 443: 213-222.
3. Xue M, Rabbani N, Thornalley P J. (2011) Glyoxalase in ageing. Seminars in Cell and Developmental Biology 22: 293-301.
4. Thornalley P J, Rabbani N. (2011) Glyoxalase in tumourigenesis and multidrug resistance. Seminars in Cell & Developmental Biology 22: 318-325.
5. Sasaki E, Kojima H, Nishimatsu H, Urano Y, Kikuchi K, Hirata Y, Nagano T. (2005) Highly Sensitive Near-Infrared Fluorescent Probes for Nitric Oxide and Their Application to Isolated Organs. Journal of the American Chemical Society 127: 3684-3685.
6. Kojima H, Sakurai K, Kikuchi K, Kawahara S, Kirino Y, Nagoshi H, Hirata Y, Nagano T. (1998) Development of a fluorescent indicator for nitric oxide based on the fluorescein chromophore. Chem Pharm Bull 46: 373-375.
7. Kojima H, Hirotani M, Nakatsubo N, Kikuchi K, Urano Y, Higuchi T, Hirata Y, Nagano T. (2001) Bioimaging of Nitric Oxide with Fluorescent Indicators Based on the Rhodamine Chromophore. Analytical Chemistry 73: 1967-1973.
8. Gabe Y, Urano Y, Kikuchi K, Kojima H, Nagano T. (2004) Highly Sensitive Fluorescence Probes for Nitric Oxide Based on Boron Dipyrromethene ChromophoreRational Design of Potentially Useful Bioimaging Fluorescence Probe. Journal of the American Chemical Society 126: 3357-3367.
9. Zhang X, Kim W-S, Hatcher N, Potgieter K, Moroz L L, Gillette R, Sweedler J V. (2002) Interfering with nitric oxide measurements. J Biol Chem 277: 48472-43478.
10. Konrade I. et al; Diabetologia 49: 662,2006.

1. A method for the detection of α,β-dicarbonyl compounds in a sample comprising the steps:
i) forming a preparation comprising (a) a fluorogenic probe comprising a 1,2 diaminophenyl moiety, the probe being stable under ambient conditions, and (b) a sample to be tested for the presence of one or more dicarbonyl groups;
ii) reacting the preparation to provide a derivatized detectable fluorescent product; and
iii) detecting the fluorescent product;
wherein the method is carried out at from pH 4-8.

2. The method according to claim 1, wherein the fluorogenic probe has at least a 20-fold increase in quantum yield on derivatisation with methylglyoxal.

3. The method according to claim 1 or 2 wherein the fluorogenic probe comprises a diaminofuorescein (DAF) diaminorhodamine (DAR), diaminophenyl boron dipyrromethane (BODIPY) or diaminocyanine moiety or a functional derivative thereof.

4. The method according to claim 3 wherein said fluorogenic probe is 4,5-diaminofluorescein, 4,5-diaminorhodamine, 8-(3,4-diaminophenyl)-2,6-bis(2-carboxyethyl)-4,4-difluoro-1,3,5,7-tetramethyl-4-bora-3a,4a-diaza-s-indacene, o-diaminocyanine, or a functional derivative thereof.

5. The method according to any one of the preceding claims wherein said α,β-dicarbonyl compound is a α-oxoaldehyde compound.

6. The method according to claim 5 wherein said α-oxoaldehyde compound is glyoxal, methylglyoxal, hydroxypyruvaldehyde, erythrosone, 3-deoxyerythrosone, ribosone, 3-deoxyribosone, glucosone, 3-deoxyglucosone or a stereoisomer thereof.

7. The method according to any one of the preceding claims wherein said sample is a biological sample.

8. The method according to claim 7 wherein said biological sample is obtained from a subject and is an isolated bodily fluid.

9. The method according to 8 wherein said bodily fluid is selected from the group consisting of: whole blood, plasma, serum, seminal fluid, urine, lymph fluid, cerebrospinal fluid, synovial fluid tears, sweat, amniotic fluid, saliva and expelled breath.

10. The method according to claim 7 wherein said biological sample comprises a cell or tissue.

11. The method according to claim 10 wherein said cell sample comprises a cell-line.

12. The method according to claim 10 wherein said cell or tissue sample is obtained from a subject.

13. The method according to any one of claims of claims 8 to 12 wherein said sample is isolated from a mammalian, preferably human.

14. The method according to any one of the preceding claims wherein (1) said method is a method of diagnosis of a subject which has or is suspected of having a disease or condition associated with elevated levels of a α,β-dicarbonyl compound; or (2) the method is carried out on a sample obtained form a subject who has or is suspected of having a disease or condition associated with elevated levels of α,β-dicarbonyl compound.

15. The method according to claim 14 wherein said disease is type 1 or type 2 diabetes.

16. The method according to claim 14 wherein said condition is a diabetic associated condition.

17. The method according to claim 16 wherein said diabetic associated condition is selected from the group: kidney disease, retinal disease, disease of peripheral nerve, cardiovascular disease and stroke, or cataract.

18. The method according to claim 14 wherein said disease is atherosclerosis.

19. The method according to claim 14 wherein said disease is hypertension or cardiovascular disease.

20. The method according to claim 14 wherein said disease is rheumatoid arthritis or osteoarthritis.

21. The method according to claim 14 wherein said disease is obesity.

22. The method according to claim 14 wherein said disease or condition is pathologic anxiety, schizophrenia, Parkinson's disease or Alzheimer's disease.

23. The method according to claim 14 wherein said disease or condition is inflammation associated with septicaemia, burns, wounding or post-surgery trauma.

24. The method according to claim 14 wherein said disease or condition is infertility, pre-eclampsia or other reproductive disorder.

25. The method according to claim 14 wherein said condition is ageing.

26. The method according to claim 14 wherein said sample is a clinical dialysis fluid or other thermally sterilised fluid with sugar solutes.

27. The method according to any one of claims 1 to 6 wherein said sample is a food or drink product, particularly but non-exclusively thermally processed products.

28. A cell based screening method to determine whether a test agent modulates the concentration of α,β-dicarbonyl containing compounds comprising the steps:
i) forming a cell culture preparation comprising a cell and an agent to be tested and optionally a fluorogenic probe as defined in any one of claims 1 to 4;
ii) culturing said cell culture in the presence of the test agent; and
iii) testing for the presence of α,β-dicarbonyl compounds in said cell or cell culture medium by addition of a fluorogenic probe as defined in any one of claims 1 to 4 to form a detectable fluorescent product and detection of said fluorescent product.

29. The method according to claim 28 wherein said α,β-dicarbonyl containing compound is as defined in claim 5 or 6.

30. The method according to claim 28 or 29 wherein said modulation is the decrease of α,β-dicarbonyl compound concentration.

31. The method according to claim 28 or 29 wherein said modulation is the increase of α,β-dicarbonyl compound concentration.

32. The method according to any one of claims 28 to 31 wherein said cell based screening assay comprises a plurality of cell based assays contained in a cell culture vessel adapted for high through put screening.

33. The method according to any one of claims 28 to 32 wherein said cell based screening assay includes an inhibitor wherein said inhibitor prevents or reduces the derivatisation of nitric oxide by 4,5-diaminofluorescein, 4,5-diaminorhodamine, diaminophenyl)-2,6-bis(2-carboxyethyl)-4,4-difluoro-1,3,5,7-tetramethyl-4-bora-3a,4a-diaza-s-indacene, o-diaminocyanine or a functional derivative thereof in said cell/tissue sample.

34. The method according to any one of claims 28 to 33 wherein said screening method includes the steps of: collating the activity data in (iii) above; converting the collated data into a data analysable form; and optionally providing an output for the analysed data.

35. The method according to any one of the preceding claims wherein said fluorescent product is detected by excitation at between 435-445 nm and emission between 500-550 nm for 4,5-diaminofluorescein and similar appropriate wavelengths for 4,5-diaminorhodamine, 8-(3,4-diaminophenyl)-2,6-bis(2-carboxyethyl)-4,4-difluoro-1,3,5,7-tetramethyl-4-bora-3a,4a-diaza-s-indacene, o-diaminocyanine or a functional derivative thereof.

36. The method according to claim 35 wherein said fluorescent product is detected by excitation at about 441 nm and emission at about 533 nm for 4,5-diaminofluorescein and similar appropriate wavelengths of emission and excitation maxima of dicarvobyla adducts for 4,5-diaminorhodamine, 8-(3,4-diaminophenyl)-2,6-bis(2-carboxyethyl)-4,4-difluoro-1,3,5,7-tetramethyl-4-bora-3a,4a-diaza-s-indacene, o-diaminocyanine or a functional derivative thereof.

37. The use of a fluorogenic probe as defined in any one of claims 1 to 4 in the detection of α,β-dicarbonyl containing compounds, wherein (a) said use is at a pH of from 4 to 8; and/or (b) said use is in a biological sample or clinical dialysis fluid, in particular a cell or tissue sample.

38. Use according to claim 37 wherein said α,β-dicarbonyl compound is as defined in claim 5 or claim 6.

39. Use according to claim 38 wherein said α,β-dicarbonyl compound is methylglyoxal.

40. A method for the detection of α,β-dicarbonyl compounds in a sample comprising the steps:
i) forming a preparation comprising (a) a fluorogenic probe as defined in any one of claims 1 to 4, and (b) a sample to be tested for the presence of one or more dicarbonyl groups;
ii) reacting the preparation to provide a derivatized detectable fluorescent product; and
iii) detection of the fluorescent product;
wherein the sample is a biological sample or clinical dialysis fluid, in particular in a cell or tissue sample.
